(19) United States
(12) Patent Application Publication
(10) Pub. No.: US 2014/0323831 A1
(43) Pub. Date: Oct. 30, 2014

(54) MEASUREMENT DEVICE, MEASUREMENT METHOD, PROGRAM, AND RECORDING MEDIUM

(71) Applicant: Sony Corporation, Tokyo (JP)

(72) Inventor: Hideo Sato, Tokyo (JP)

(73) Assignee: SONY CORPORATION, Tokyo (JP)

(21) Appl. No.: 14/364,542

(22) PCT Filed: Dec. 3, 2012
(86) PCT No.: PCT/JP2012/081255

§ 371 Date: Jun. 11, 2014

(30) Foreign Application Priority Data
Dec. 19, 2011 (JP) 2011-277614

(51) Int. Cl. A61B 5/1455; A61B 5/00

(52) U.S. Cl. CPC A61B 5/1455; A61B 5/0059; A61B 5/489
USPC 600/301; 600/322

(57) ABSTRACT

Provided is a measurement device including a measurement unit to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape, a vein position specification unit to specify a position of a vein present inside the living body based on the captured image, a vein depth specification unit to specify a depth of the vein based on the captured image, and a blood component estimation unit configured to estimate a blood component of the vein based on information obtained from the detected measurement light using the position and the depth of the vein.

### TECHNICAL FIELD

The present disclosure relates to a measurement device, a measurement method, a program, and a recording medium.

### BACKGROUND ART

Recently, technologies for measuring a biological substance in a non-invasive manner through optical measurement have been developed. The technologies have been used in, for example, measurement of a component included in a hypodermal tissue or arterial blood. On the other hand, as disclosed in Patent Literature 1, for example, the technology for recognizing a pattern of a vein under skin through optical measurement is also known.

### CITATION LIST

### Patent Literature

Patent Literature 1: JP 2007-72677A

### SUMMARY OF INVENTION

### Technical Problem

However, in such a technology, it is difficult to measure a blood component of a vein. This is because, when measurement of venous blood is attempted through optical measurement, influence of arterial blood and a hypodermal tissue is substantial and thus it is difficult to separate the influence. However, in measurement of a body fluid of a hypodermal tissue in which a concentration of a component is shown differently from in blood or measurement of arterial blood that shows a considerable temporal change in a concentration of a component caused by having a meal or the like, accurate analysis of a blood component is difficult.

Thus, the present disclosure proposes a novel and improved measurement device, measurement method, program, and recording medium which enable accurate measurement of a blood component of a vein through optical measurement.

### Solution to Problem

According to the present disclosure, there is provided a measurement device including a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape, a vein position specification unit configured to specify a position of a vein present inside the living body based on the captured image, a vein depth specification unit configured to specify a depth of the vein based on the captured image, and a blood component estimation unit configured to estimate a blood component of the vein based on information obtained from the detected measurement light using the position of the vein and the depth of the vein.

According to the present disclosure, there is provided a measurement method including radiating measurement light having a predetermined wavelength to at least a part of a living body, detecting the measurement light scattered inside the living body and then discharged from a surface of the living body, and acquiring a captured image of the living body by collecting the discharged measurement light using a lens array having a plurality of light receiving lenses arranged in an array shape, specifying a position of a vein present inside the living body based on the captured image, specifying a depth of the vein based on the captured image, and estimating a blood component of the vein based on information obtained from the detected measurement light using the position of the vein and the depth of the vein.

According to the present disclosure, there is provided a program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute a vein position specification function of specifying a position of a vein present inside the living body based on the captured image, a vein depth specification function of specifying a depth of the vein based on the captured image, and a blood component estimation function of estimating a blood component of the vein based on information obtained from the detected measurement light using the position of the vein and the depth of the vein.

According to the present disclosure, there is provided a computer-readable recording medium on which a program is recorded, the program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute a vein position specification function of specifying a position of a vein present inside the living body based on the captured image, a vein depth specification function of specifying a depth of the vein based on the captured image, and a blood component estimation function of estimating a blood component of the vein based on information obtained from the detected measurement light using the position of the vein and the depth of the vein.

According to the present disclosure, there is provided a measurement device including a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape, a vein position specification unit configured to specify a position of a vein present inside the living body based on the captured image, a vein depth specification unit configured to specify a depth of the vein based on the captured image, and a blood component estimation unit configured to estimate a thickness of a body tissue present between a surface of the living body and the vein based on the depth of the vein, to exclude influence of the body tissue of the estimated thickness from information obtained from the measurement light detected in the position of the vein, to extract a time change component derived from beats of an artery present inside the living body from information obtained from the measurement light detected in an adjacent area adjacent to the position of the vein, and to estimate a blood component of the vein by further eliminating the time change component from the information obtained from the measurement light detected in the position of the vein.

According to the present disclosure, there is provided a measurement method including radiating measurement light having a predetermined wavelength to at least a part of a living body, detecting the measurement light scattered inside the living body and then discharged from a surface of the living body, and acquiring a captured image of the living body by collecting the discharged measurement light using a lens array having a plurality of light receiving lenses arranged in an array shape, specifying a position of a vein present inside the living body based on the captured image, specifying a depth of the vein based on the captured image, and estimating a thickness of a body tissue present between a surface of the living body and the vein based on the depth of the vein, excluding influence of the body tissue of the estimated thickness from information obtained from the measurement light detected in the position of the vein, extracting a time change component derived from beats of an artery present inside the living body from information obtained from the measurement light detected in an adjacent area adjacent to the position of the vein, and estimating a blood component of the vein by further eliminating the time change component from the information obtained from the measurement light detected in the position of the vein.

According to the present disclosure, there is provided a program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute a vein position specification function of specifying a position of a vein present inside the living body based on the captured image, a vein depth specification function of specifying a depth of the vein based on the captured image, and a blood component estimation function of estimating a thickness of a body tissue present between a surface of the living body and the vein based on the depth of the vein, excluding influence of the body tissue of the estimated thickness from information obtained from the measurement light detected in the position of the vein, extracting a time change component derived from beats of an artery present inside the living body from information obtained from the measurement light detected in an adjacent area adjacent to the position of the vein, and thereby estimating a blood component of the vein by further eliminating the time change component from the information obtained from the measurement light detected in the position of the vein.

According to the configurations described above, by specifying a position and a depth of a vein that is a measurement target present inside a living body, noise caused by, for example, influence of a body component and influence of an artery can be separated from an optical spectrum of measurement light detected in a region of a vein, and thereby a blood component of the vein can be accurately measured.

### Advantageous Effects of Invention

According to the present disclosure described above, it is possible to accurately measure a blood component of a vein through optical measurement.

### BRIEF DESCRIPTION OF DRAWINGS

FIG. 1 is a block diagram showing an overall configuration of a measurement device according to a first embodiment of the present disclosure.

FIG. 2 is a diagram showing a configuration of a measurement unit of the measurement device according to the first embodiment of the present disclosure.

FIG. 3 is a diagram for describing information obtained from measurement light in the first embodiment of the present disclosure.

FIG. 4 is a diagram for describing a process of a vein position specification unit in the first embodiment of the present disclosure.

FIG. 5 is a diagram for describing a process of a vein depth specification unit in the first embodiment of the present disclosure.

FIG. 6 is a diagram for describing a process of a blood component estimation unit in the first embodiment of the present disclosure.

FIG. 7 is a graph for describing the process of the blood component estimation unit in the first embodiment of the present disclosure.

FIG. 8 is a flowchart showing a process performed in the first embodiment of the present disclosure.

FIG. 9 is a diagram showing a configuration of a measurement unit of a measurement device according to a second embodiment of the present disclosure.

FIG. 10 is a diagram showing a configuration of a measurement unit of a measurement device according to a third embodiment of the present disclosure.

FIG. 11 is a diagram showing a configuration of a measurement unit of a measurement device according to a fourth embodiment of the present disclosure.

FIG. 12 is a diagram showing a configuration of a measurement unit of a measurement device according to a fifth embodiment of the present disclosure.

FIG. 13 is a diagram showing a configuration of a measurement unit of a measurement device according to a sixth embodiment of the present disclosure.

FIG. 14 is a block diagram for describing a hardware configuration of an information processing apparatus according to an embodiment of the present disclosure.

### DESCRIPTION OF EMBODIMENTS

Hereinafter, preferred embodiments of the present invention will be described in detail with reference to the appended drawings. Note that, in this specification and the drawings, elements that have substantially the same function and structure are denoted with the same reference signs, and repeated explanation is omitted.

Note that description will be provided in the following order.

1. First embodiment
1-1. Overall configuration of a measurement device
1-2. Configuration of a measurement unit
1-3. Process of a vein position specification unit
1-4. Process of a vein depth specification unit
1-5. Process of a blood component estimation unit
1-6. Processing flow

2. Second embodiment

3. Third embodiment

4. Fourth embodiment

5. Fifth embodiment

6. Sixth embodiment

7. Supplement

### 1. First Embodiment

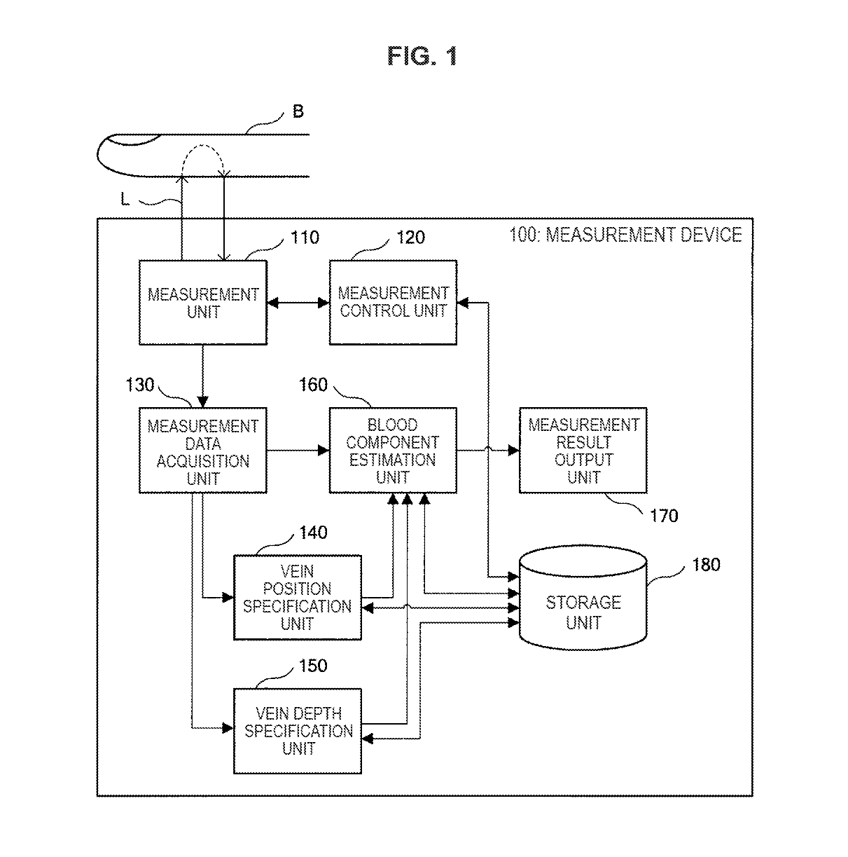

First, a first embodiment of the present disclosure will be described with reference to FIGS. 1 to 8.

### (1-1. Overall Configuration of a Measurement Device)

FIG. 1 is a block diagram showing an overall configuration of a measurement device according to the first embodiment of the present disclosure. Referring to FIG. 1, the measurement device 100 includes a measurement unit 110, a measurement control unit 120, a measurement data acquisition unit 130, a vein position specification unit 140, a vein depth specification unit 150, a blood component estimation unit 160, a measurement result output unit 170, and a storage unit 180.

The measurement unit 110 functions as a measurement probe that measures at least one part of a living body B. The measurement unit 110 radiates measurement light L having a predetermined wavelength to at least one part of the living body B to detect the measurement light L scattered inside the living body B and discharged from a surface of the living body B and thereby to acquire a captured image of the living body B by collecting the discharged measurement light L using a lens array having a plurality of light receiving lenses arranged in an array shape.

For example, the measurement unit 110 radiates red light or near-infrared light having a predetermined wavelength to one part (measurement portion) of the living body B as the measurement light L to capture an image of the skin of the measurement portion. In addition, the measurement unit 110 detects the measurement light L scattered inside the living body B. In this case, since part of the measurement light L is absorbed by arteries, veins, and other body tissues, an amount of the light is reduced in comparison to that at the time of radiation. For example, the measurement unit 110 measures distribution of a light amount of the measurement light L discharged from the surface of the living body B to set measurement data relating to the measurement portion. The measurement data is an example of information obtained from the measurement light detected by the measurement unit 110.

As will be described below, in the present embodiment, a blood component of a vein present inside the living body B is estimated through optical measurement. A technique of the optical measurement may use a light-absorbing characteristic of a substance in a body as described above, or may use a scattering characteristic or an optical rotation characteristic thereof. The measurement unit 110 radiates light having an appropriate wavelength as the measurement light L according to a technique used in the measurement device 100. For example, when an amount of glucose included in venous blood is measured using the light-absorbing characteristic described above, for example, the measurement unit 110 radiates near infrared light having a wavelength of 1400 nm to 2200 nm as the measurement light L. Note that the measurement light L radiated by the measurement unit 110 is not limited to light having a single wavelength. For example, the measurement unit 110 may radiate a plurality of light beams each having different wavelengths as the measurement light L in a time division manner. In this case, the measurement unit 110 may use different light beams in each of acquisition of a captured image and measurement of distribution of a light amount.

The measurement control unit 120 is realized by, for example, a CPU (Central Processing Unit), a ROM (Read Only Memory), a RAM (Random Access Memory), a communication device, and the like. The measurement control unit 120 controls driving of a light source unit, an image sensor, and the like included in the measurement unit 110, and controls overall measurement processes of the living body B performed in the measurement unit 110. For example, the measurement control unit 120 controls a scanning timing of the image sensor, selection of a pixel, and the like based on a predetermined synchronization signal. In addition, the measurement control unit 120 controls a radiation timing of the measurement light L and a light amount of the light source unit, and the like. In order to perform the control, the measurement control unit 120 may refer to various programs, parameters, databases, or the like recorded in the storage unit 180 or the like.

The measurement data acquisition unit 130 is realized by, for example, a CPU, a ROM, a RAM, a communication device, and the like. The measurement data acquisition unit 130 acquires the captured image and measurement data acquired by the measurement unit 110 and then outputs them to the vein position specification unit 140, the vein depth specification unit 150, the blood component estimation unit 160. The measurement data acquisition unit 130 may acquire the measurement data in a time series manner by sequentially acquiring the measurement data output from the measurement unit 110 at each predetermined timing. The measurement data acquisition unit 130 may store the acquired measurement data in the storage unit 180 as history information in association with time information relating to dates, times, and the like when the data is acquired.

The vein position specification unit 140 is realized by, for example, a CPU, a ROM, a RAM, and the like. The vein position specification unit 140 specifies a position of a vein present inside the living body B based on the captured image acquired from the measurement unit 110 via the measurement data acquisition unit 130. Note that details of a process of the vein position specification unit 140 will be described below.

The vein depth specification unit 150 is realized by, for example, a CPU, a ROM, a RAM, and the like. The vein depth specification unit 150 specifies a depth of a vein present inside the living body B based on the captured image acquired from the measurement unit 110 via the measurement data acquisition unit 130. Note that details of a process of the vein depth specification unit 150 will be described below.

The blood component estimation unit 160 is realized by, for example, a CPU, a ROM, a RAM, and the like. The blood component estimation unit 160 estimates a blood component of the vein present inside the living body B based on the measurement data acquired from the measurement unit 110 via the measurement data acquisition unit 130. The blood component estimation unit 160 uses the position of the vein specified by the vein position specification unit 140 and the depth of the vein specified by the vein depth specification unit 160 in estimation of the blood component. The blood component estimation unit 160 may further have a result of the estimation go through calibration based on information of the blood component of the vein measured in advance through blood sampling. The blood component estimation unit 160 may store the estimated data of the blood component in the storage unit 180 as history information in association with time information relating to dates and times when the data is acquired. Note that details of a process of the blood component estimation unit 160 will be described below.

The measurement result output unit 170 is realized by, for example, a CPU, a ROM, a RAM, an output device, a communication device, and the like. The measurement result output unit 170 outputs information relating to the blood component of the vein present inside the living body B estimated by the blood component estimation unit 160. The measurement result output unit 170 may output the information to the output device such as a display that the measurement device 100 has or may output the information on a paper medium using a printer, or the like. In addition, the measurement result output unit 170 may output the information relating to the blood component of the vein to a display, various kinds of information processing apparatuses, and the like provided outside the measurement device 100. In this manner, as the measurement result output unit 170 outputs the information relating to the blood component of the vein, a user of the measurement device 100 can ascertain a measurement result.

The storage unit 180 is realized by, for example, a RAM that the measurement device 100 has, a storage device, and the like. In the storage unit 180, the measurement data measured by the measurement unit 110, the captured image, and various programs, parameters, data, and the like used in processes executed by the measurement device 100 may be recorded. In addition, in the storage unit 180, intermediate data such as variables generated when the measurement device 100 executes processes may be recorded, in addition to the data. Each of the processing units including the measurement control unit 120, the measurement data acquisition unit 130, the vein position specification unit 140, the vein depth specification unit 150, the blood component estimation unit 160, the measurement result output unit 170, and the like may be able to freely access the storage unit 180 to write or read the data thereon or therefrom.

Note that the measurement control unit 120, the measurement data acquisition unit 130, the vein position specification unit 140, the vein depth specification unit 150, the blood component estimation unit 160, and the measurement result output unit 170 described above may be realized as a part of the measurement device 100, or may be realized by an external device such as a computer connected to the measurement device 100. In addition, measurement data generated by the measurement unit 110 may be analyzed by storing the measurement data in a removable storage medium or the like, and detaching the storage medium from the measurement device 100 to be connected to another device that has the measurement control unit 120, the measurement data acquisition unit 130, the vein position specification unit 140, the vein depth specification unit 150, the blood component estimation unit 160, and the measurement result output unit 170.

In addition, as another embodiment of the present disclosure, a computer program for realizing each of the functions of the measurement device according to the present embodiment as described above may be produced and installed in a personal computer or the like. A computer-readable recording medium in which such a computer program is stored may be provided. The recording medium can be, for example, a magnetic disk, an optical disc, a magneto-optical disc, a flash memory, or the like. In addition, the computer program may be distributed via, for example, a network without using such a recording medium.

### (1-2. Configuration of the Measurement Unit)

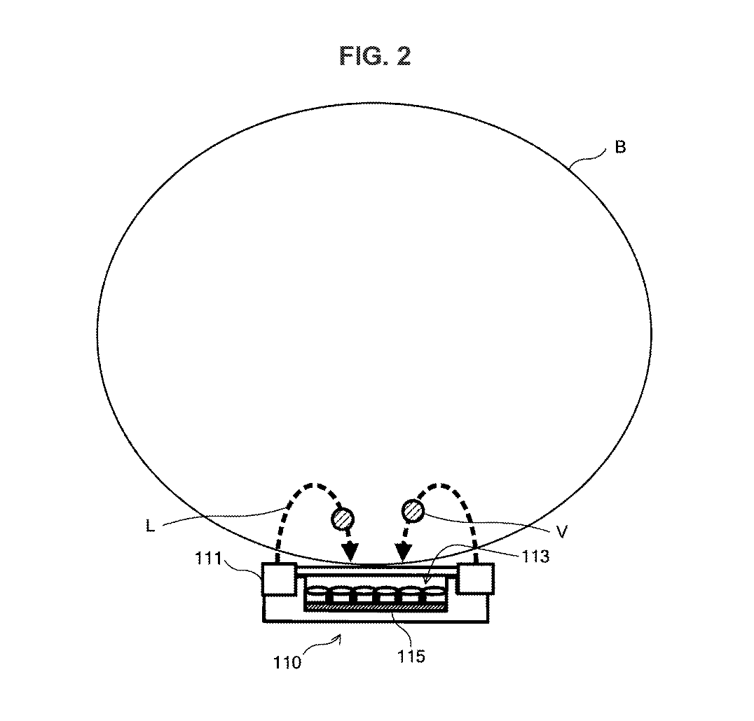

FIG. 2 is a diagram showing a configuration of the measurement unit of the measurement device according to the first embodiment of the present disclosure. Referring to FIG. 2, the measurement unit 110 includes a light source unit 111, a microlens array 113, and an image sensor 115.

The light source unit 111 radiates the measurement light L having a predetermined wavelength to the living body B. In the present embodiment, the light source unit 111 is provided adjacent to the microlens array 113 so that an emission surface of the measurement light L faces the living body B. The light source unit 111 may be provided at an edge of the microlens array 113. As the light source unit 111, for example, a light emitting diode (LED: Light Emitting Diode), a small-sized laser, or the like is used. As described above, the light source unit 111 may radiate light having a single wavelength as the measurement light L or may radiate a plurality of light beams having different wavelengths in a time division manner.

The microlens array (MLA: Micro Lens Array) 113 guides the measurement light L reflected and spread inside the living body B and then discharged from a surface of the living body B to the image sensor 115. The microlens array 112 includes a plurality of light receiving lenses disposed in, for example, a grid shape and each of the light receiving lenses guides the measurement light L discharged from the surface of the living body B of a predetermined region to a predetermined light receiving element of the image sensor 115. Since the microlens array 113 is a lens array having little curvature of field and no distortion in a depth direction, by guiding the measurement light L to the image sensor 115 using the microlens array 113, satisfactory measurement data can be obtained.

The image sensor 115 converts intensity of the measurement light L sensed by a photodetector (Photo Detector: PD) into an electric signal and then outputs the signal to the measurement data acquisition unit 130. As the image sensor 115, for example, a CCD (Charge Coupled Devices) type image sensor, a CMOS (Complementary Metal Oxide Semiconductor) type image sensor, a sensor having a light receiving element of an organic EL, or a two-dimensional area sensor such as a TFT (Thin Film Transistor) type image sensor is used. In the present embodiment, both of the captured image for specifying a position and a depth of a vein that will be described later and the distribution of a light amount for estimating the blood component are acquired by the image sensor 115. Thus, it is desirable for the image sensor 115 to deal with light having a wide wavelength band. Note that, when light beams having different wavelengths are radiated from the light source unit 111 in a time division manner, the image sensor 115 may acquire the captured image and the distribution of the light amount respectively using light beams having different wavelengths.

Here, at least part of the measurement light L, which has been radiated by the light source unit 111 and reflected and spread inside the living body B as shown in the drawing, passes through a vein V present inside the living body B. At this time, a change occurs in the measurement light L due to a characteristic of a blood component of the vein V. The measurement device 100 measures the change of the measurement light L using the microlens array 113 and the image sensor 115 and then specifies the blood component of the vein V. As described above, the characteristic of the blood component used here may be the light-absorbing characteristic, or may be the scattering characteristic, the optical rotation characteristic, or the like. The measurement unit 110 appropriately includes other constituent elements according to such a characteristic used here. For example, when the optical rotation characteristic of the blood component is used, a polarization filter may be provided between the microlens array 113 and the living body B.

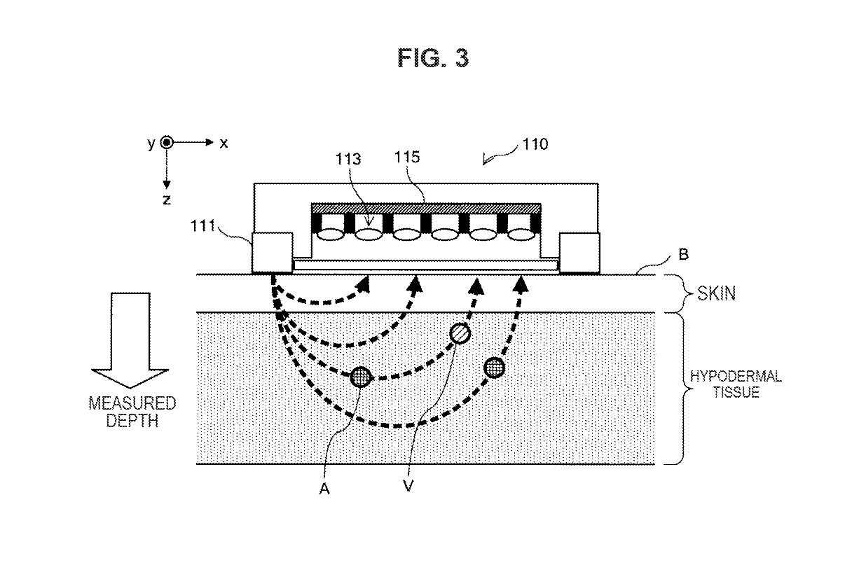

FIG. 3 is a diagram for describing information obtained from measurement light in the first embodiment of the present disclosure. Since the living body B is a medium that causes light to be scattered extremely well, the measurement light L radiated from the light source unit 110 moves forward while being spread inside the living body B, is discharged from the surface of the living body B at a certain position and then incident on the microlens array 113, and then is measured by the image sensor 115.

At this time, as the position of the light receiving lenses of the microlens array 113 becomes farther from the light source unit 111, the measurement light L that has been scattered to a deeper position of the living body B and then returned to a surface of the body is detected. In other words, a light receiving lens of which the position in the x axis direction in the drawing is set further away from the light source unit 111 detects the measurement light L that has permeated to a deeper position. In the present embodiment, the blood component estimation unit 160 of the measurement device 100 estimates an optical path of the measurement light L by, for example, modeling a characteristic such as scattering or attenuation of the light at a position of each light receiving lens of the microlens array 113 so as to be used in estimation of the blood component.

The measurement light L is affected in the course of scattering as described above by absorption of energy of a specific wavelength, occurrence of polarization, or scattering due to a body component such as a blood vessel, skin, or a hypodermal tissue located on the optical path. As shown in the drawing, the measurement light L radiated from the light source unit 111 and incident on the microlens array 113 is affected by not only the vein V located on the optical path but also an artery A present inside the living body B and a body tissue such as skin or a hypodermal tissue. Thus, in order to accurately estimate the blood component of the vein V, it is desirable to exclude such influence of other components including the artery A and body tissue from a measurement result.

To this end, in the present embodiment, specification of a position of the vein V by the vein position specification unit 140 and specification of a depth of the vein V by the vein depth specification unit 150 are executed. Note that, in description of the present embodiment, a “position” corresponds to a position in the direction parallel to the measurement unit 110, in other words, coordinates of the x axis and y axis of the drawing, and a “depth” corresponds to a depth in the direction perpendicular to the measurement unit 110, in other words, a coordinate of the z axis of the drawing.

### (1-3. Process of the Vein Position Specification Unit)

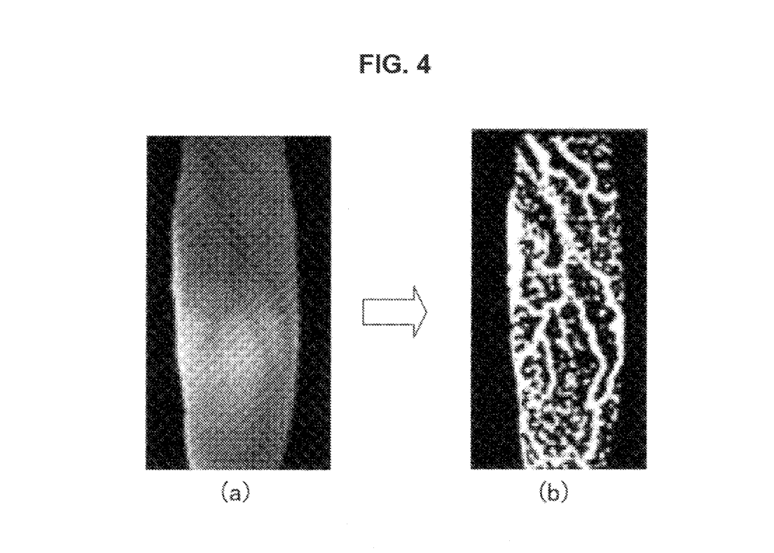

FIG. 4 is a diagram for describing a process of the vein position specification unit in first embodiment of the present disclosure. Referring to FIG. 4, the vein position specification unit 140 performs image processing on a captured image acquired by the measurement unit 110 as shown in (a) and then generates information indicating a position of the vein V as shown in (b).

The measurement light L emitted from the light source unit 111 of the measurement unit 110 and incident on the inside of the living body B is scattered inside the living body B and then incident on the microlens array 113. At this moment, in the position of the vein V inside the living body B, light absorption occurs due to venous blood and accordingly intensity of the measurement light L discharged from the living body B is lowered. Thus, in the captured image acquired by the measurement unit 110, the position corresponding to the vein V is the region having lower luminance than peripheries. Note that, hereinbelow, a shape of the vein V expressed in a captured image is also referred to as a vein pattern.

The vein position specification unit 140 extracts a vein pattern by applying, for example, a difference filter to the captured image. The difference filter is a filter that outputs, with respect to a pixel of interest and a peripheral pixel thereof, a higher value of a portion having a great difference between the pixel of interest and the peripheral pixel as an output value. In other words, the difference filter is a filter that emphasizes a line or a border in an image through an arithmetic operation using the difference between grayscale values of the pixel of interest and a pixel nearby.

In general, when a filtering process is executed on image data u(x, y) having a grid point (x, y) of a two-dimensional plane as a variable using a filter h(x, y), image data v(x, y) is generated as shown in Formula 101 below. Note that, in Formula 101 below, “*” denotes convolution.

$$\begin{matrix}[ Math . 1 ] & \\ v ( x , y ) = u ( x , y ) * h ( x , y ) = {\sum }_{{m}_{1}}^{ } {\sum }_{{m}_{2}}^{ } h ( {m}_{1} , {m}_{2} ) u ( x - {m}_{1} , y - {m}_{2} ) = {\sum }_{{m}_{1}}^{ } {\sum }_{{m}_{2}}^{ } u ( {m}_{1} , {m}_{2} ) h ( x - {m}_{1} , y - {m}_{2} ) & ( Formula 101 )\end{matrix}$$

In the extraction of the vein pattern according to the present embodiment, a differential filter such as a first-order space differential filter or a second-order space differential filter may be used as the difference filter. The first-order space differential filter is a filter that computes the difference of grayscale values of pixels adjacent to each other in a lateral direction and a longitudinal direction with respect to a pixel of interest, and the second-order space differential filter is a filter that extracts a portion having a large change amount of the difference between grayscale values with respect to the pixel of interest.

As the second-order space differential filter described above, for example, a Log (Laplacian of Gaussian) filter shown below can be used. The Log filter (Formula 103) is expressed by secondary differentiation of a Gaussian filter (Formula 102) that is a smoothing filter using a Gaussian function. Here, in Formula 102 below, σ denotes a standard deviation of the Gaussian function and is a variable indicating a degree of smoothing of the Gaussian filter. In addition, σ in Formula 103 below is a parameter indicating the standard deviation of the Gaussian function as in Formula 102, and by changing the value of σ, it is possible to change an output value obtained when a Log filtering process is performed.

$$\begin{matrix}[ Math . 2 ] & \\ {h}_{gauss} ( x , y ) = \frac{1}{2 {πσ}^{2}} exp \{ \frac{- ( {x}^{2} + {y}^{2} )}{2 {σ}^{2}} \} & ( Formula 102 ) \\ {h}_{Log} ( x , y ) = {∇}^{2} \cdot {h}_{gauss} ( x , y ) = ( \frac{{\partial }^{2}}{\partial {x}^{2}} + \frac{{\partial }^{2}}{\partial {y}^{2}} ) {h}_{gauss} = \frac{( {x}^{2} + {y}^{2} - 2 {σ}^{2} )}{2 {πσ}^{2}} exp \{ \frac{- ( {x}^{2} + {y}^{2} )}{2 {σ}^{2 }} \} & ( Formula 103 )\end{matrix}$$

The vein position specification unit 140 may execute a post-process such as a threshold value process, a binarization process, or a line-thinning process on the captured image to which the difference filter has been applied as described above. After passing through such a post-process, a skeleton of the vein pattern as shown in (b) of the drawing is extracted. The vein position specification unit 140 provides the vein pattern, for example, extracted as described above to the blood component estimation unit 160 as information indicating the position of the vein V present inside the living body.

### (1-4. Process of the Vein Depth Specification Unit)

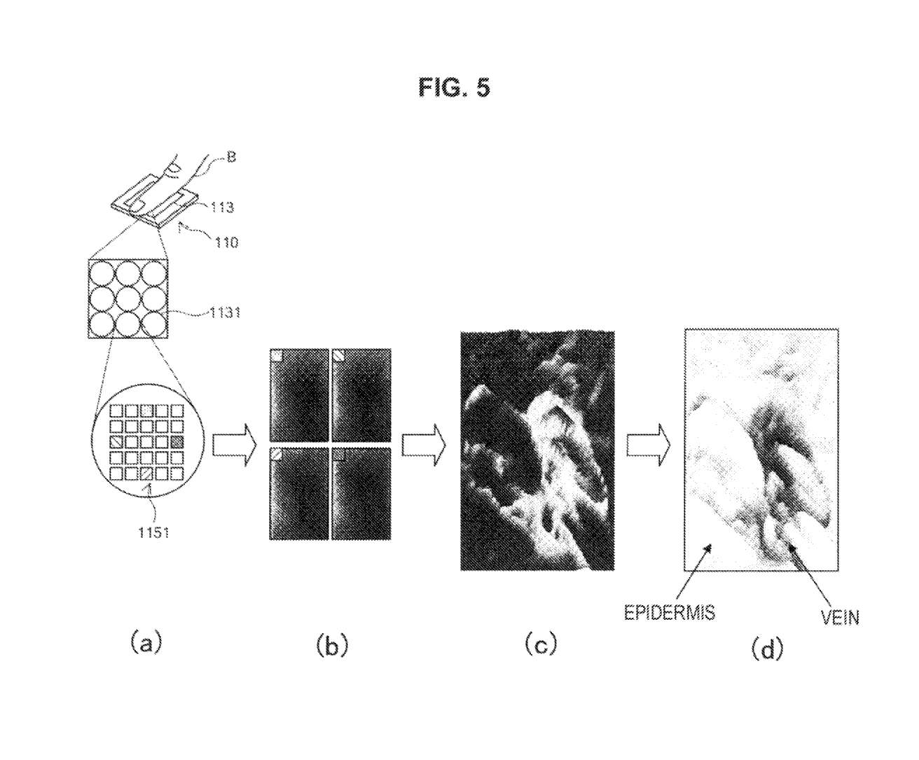

FIG. 5 is a diagram for describing a process of the vein depth specification unit in first embodiment of the present disclosure. Referring to FIG. 5, the vein depth specification unit 150 performs image-processing on the captured images as shown in (b) acquired by the measurement unit 110 by setting as shown in (a), thereby acquiring parallax information as shown in (c). Furthermore, the vein depth specification unit 150 acquires depth information of the vein V as shown in (d) based on the parallax information. In this case, the vein depth specification unit 150 may use the technique of Light Field Photography.

As shown in (a), in the measurement unit 110, measurement light discharged from a surface (body surface) of the living body B is incident on the microlens array 113. As described above, the microlens array 113 includes a plurality of light receiving lenses 1131 disposed in a grid shape or the like. Here, light guided by each of the light receiving lenses 1131 forms an image in light receiving elements 1151 on the image sensor 115, and thereby the captured images are generated by the light receiving elements 1151.

Here, as shown in the drawing, a plurality of light receiving elements 1151 are allocated to one light receiving lens 1131 in the present embodiment. A substantially identical image is formed in light receiving elements 1151 allocated to the same light receiving lens 1131, but directions of an imaging target with regard to each of the light receiving elements 1151 differ according to an arrangement position of the light receiving elements 1151. Thus, as shown in (b), parallax is included in the captured images generated by each of the light receiving elements 1151 allocated to the same light receiving lens 1131 in the longitudinal direction and lateral direction.

For example, the vein depth specification unit 150 performs differential processing on the captured images obtained from each of the light receiving elements 1151 corresponding to the same light receiving lens 1131, thereby detecting edges. The vein depth specification unit 150 detects the parallax by comparing the positions of the same point of the imaging target indicated by the edges located in each of the captured images. The parallax detected here and represented as a map is shown in (c). Note that, in the present embodiment, since the light receiving lenses 1131 are set so that the vein V inside the living body B is included in depth of field, the detected edges are attributable to the image of the vein V.

Furthermore, the vein depth specification unit 150 computes an isolation distance between the light receiving elements 1151 and the imaging target, i.e., the vein V, using the parallax between the captured images detected as described above and information relating to the positional relationship between the light receiving elements 1151 that have acquired each of the captured images. When the distance from the light receiving elements 1151 to the living body B is subtracted from the separation distance, the depth from the surface of the living body B to the vein V is computed. The map of the depth computed as above is shown in (d). In the example shown in the drawing, the portion in which the depth is detected is a region of the vein V, and the portion in which the depth is not detected is an epidermal region not including the vein V.

On the other hand, the differences in the directions of the imaging target between each of the light receiving elements 1151 corresponding to the same light receiving lens 1131 are also the differences in directions of light incident on the light receiving lens 1131. In other words, each of the light receiving elements 1151 receives light incident on the light receiving lens 1131 in different directions. When the technique of Light Field Photography introduced in JP 2008-515110T or the like is used, for example, it is possible to generate an image of which focus is on an arbitrary position by combining light beams expressed in images acquired by the light receiving elements 1151 corresponding to each of the light receiving lenses 1131 using software. Using this technology, the vein depth specification unit 150 may acquire captured images in which focus positions are arbitrarily set, and more specifically compute the depth of the vein V.

### (1-5. Process of the Blood Component Estimation Unit)

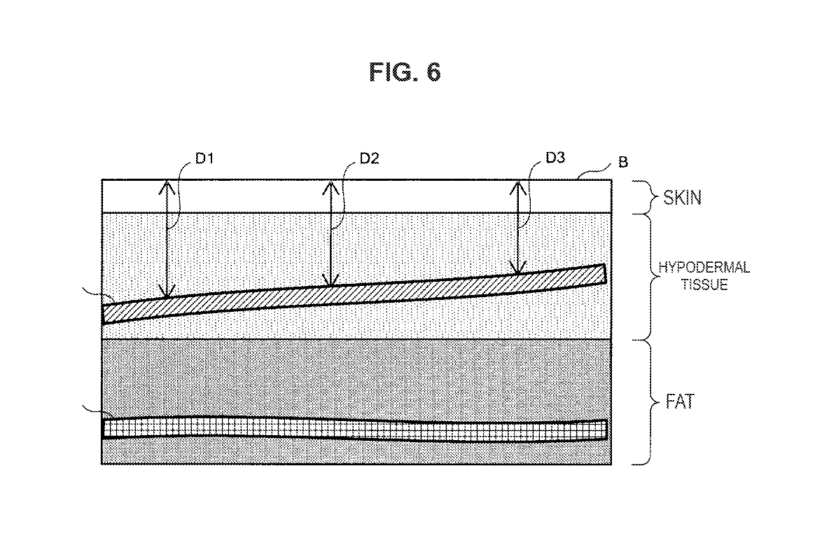

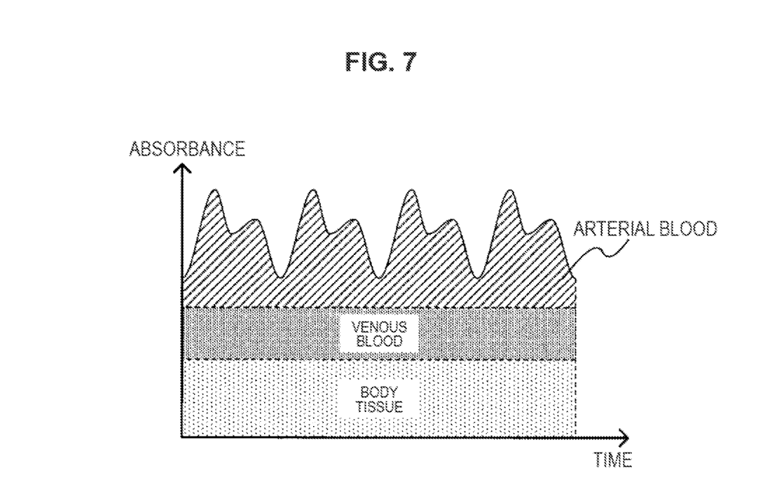

FIGS. 6 and 7 are diagrams for describing a process of the blood component estimation unit in first embodiment of the present disclosure.

The blood component estimation unit 160 analyzes an optical spectrum of the measurement light L discharged from the region corresponding to the vein V of the living body B. The region corresponding to the vein V is found based on, for example, information of the position of the vein V specified by the vein position specification unit 140. Here, as shown in FIGS. 6 and 7, in the optical spectrum of the measurement light L in the region of the vein V, a change caused by a blood component of the vein V, influence of a blood component of the artery A, influence of a component in the skin or hypodermal tissue present between the vein V and a surface of the living body B overlap. Thus, the blood component estimation unit 160 separates such influence of the components from a measurement result.

First, with regard to the influence of the skin of hypodermal tissue, the blood component estimation unit 160 separates the influence based on the information indicating the depth of the vein V acquired by the vein depth specification unit 150. As shown in the drawing, as a result of a process performed by the vein depth specification unit 150, depths of the vein V from a surface of the living body B (for example, D1 to D3) are computed. The blood component estimation unit 160 computes the influence of the skin and hypodermal tissue incorporated into the optical spectrum of the measurement light L by multiplying the depths D1 to D3, i.e., the thicknesses of the skin and hypodermal tissue, by a light absorption spectrum of the skin and hypodermal tissue measured in advance, and then removes the influence from the measurement result.

Next, with regard to the influence of the blood component of the artery A, the blood component estimation unit 160 separates the influence based on the information indicating the position of the vein V acquired by the vein position specification unit 140 and a time-series change of the optical spectrum of the measurement light L. For example, the blood component estimation unit 160 extracts a time change component that is synchronized with pulsation and is included in the optical spectrum of the measurement light L detected in a region adjacent to the position of the vein V, and then removes the vector component from the measurement result as a component derived from beats of the artery. The reason for using the optical spectrum of the measurement light L detected in the region adjacent to the position of the vein V is that the measurement light L detected in the adjacent region is considered not to have passed through the vein V and considered to be suitable as an optical spectrum showing a noise component.

Through the process described above, the blood component estimation unit 160 separates the influence of the artery A, the skin, and the hypodermal tissue from the optical spectrum of the measurement light L discharged from the region of the vein V. In the measurement device 100, by analyzing the optical spectrum from which the influence of the artery A, the skin, and the hypodermal tissue is separated, the blood component estimation unit 160 can more accurately estimate the blood component of the vein V.

### (1-6. Processing Flow)

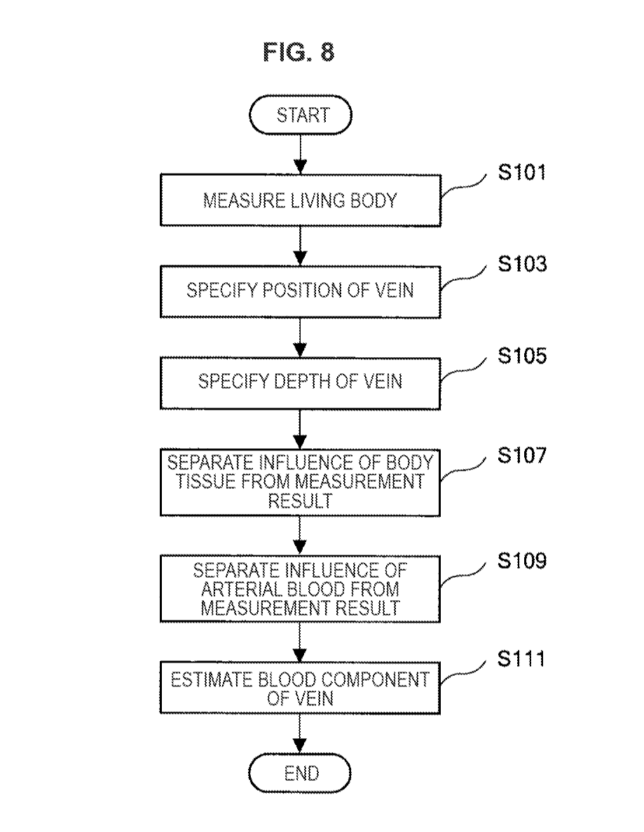

FIG. 8 is a flowchart showing the process performed in the first embodiment of the present disclosure.

First, the measurement unit 110 measures a living body (Step S101). Here, the measurement unit 110 measures the living body B in such a way that the measurement light L is radiated from the light source unit 111 to be incident on the living body B, and then the measurement light L scattered inside the living body B and then discharged from a surface of the living body B is incident on the microlens array 113 to be received by the image sensor 115.

Next, the vein position specification unit 140 specifies a position of the vein V present inside the living body B (Step S103). Here, the vein position specification unit 140 specifies the position of the vein V by, for example, applying image processing using the difference filter to captured images of the living body B acquired by the image sensor 115.

Next, the vein depth specification unit 150 specifies a depth of the vein V (Step S105). Here, the vein depth specification unit 150 specifies the depth of the vein V based on parallax extracted from the captured images each acquired by the plurality of light receiving elements 1151 of the image sensor 115 corresponding to the light receiving lens 1131 of the microlens array 113.

Next, the blood component estimation unit 160 estimates a thickness of a body tissue such as skin, a hypodermal tissue, or the like present between the vein V and the living body B based on the depth of the vein V specified by the vein depth specification unit 150, and then separates influence of the body tissue of the thickness from the optical spectrum of the measurement light L acquired by the image sensor 115 in the region of the vein V (Step S107).

Next, the blood component estimation unit 160 separates a time change component of the optical spectrum of the measurement light L that is synchronized with pulsation in a region adjacent to the position of the vein V from the optical spectrum of the measurement light L acquired by the image sensor 115 in the position of the vein V based on influence of an artery component (Step S109).

Next, the blood component estimation unit 160 estimates a blood component of the vein V based on the optical spectrum from which the influence of the artery A, the skin, and the hypodermal tissue is separated in Steps S107 and S109 (Step S111).

Note that, in the processing flow described above, Steps S103 and S105 in which the captured images acquired in Step S101 are received as inputs may be executed, for example, in parallel with each other after the execution of Step S101 or in the reverse order. In addition, in the same manner, Step S109 in which the information of the position of the vein V specified in Step S103 is used may be executed in parallel with Step S105 or Step S107 or prior to these steps.

Hereinabove, the first embodiment of the present disclosure has been described. In the present embodiment, by specifying a position and a depth of the vein V which is a measurement target present inside the living body B, influence of a body component and influence of the artery A are separated from the optical spectrum of the measurement light L detected in the region of the vein V, and thereby a blood component of the vein V can be accurately measured.

In addition, in the present embodiment, by using the optical system including the microlens array 113 and the image sensor 115 in the measurement unit 110, the measurement unit 110 can be made thin. Here, the plurality of light receiving elements 1151 of the image sensor 115 correspond to each of the light receiving lens 1131 of the microlens array 113. Specification of the depth of the vein V based on parallax information can be compatible with resolution of the captured images. In addition, by providing the light source unit 111 adjacent to the microlens array 113, the measurement unit 110 can be miniaturized.

### 2. Second Embodiment

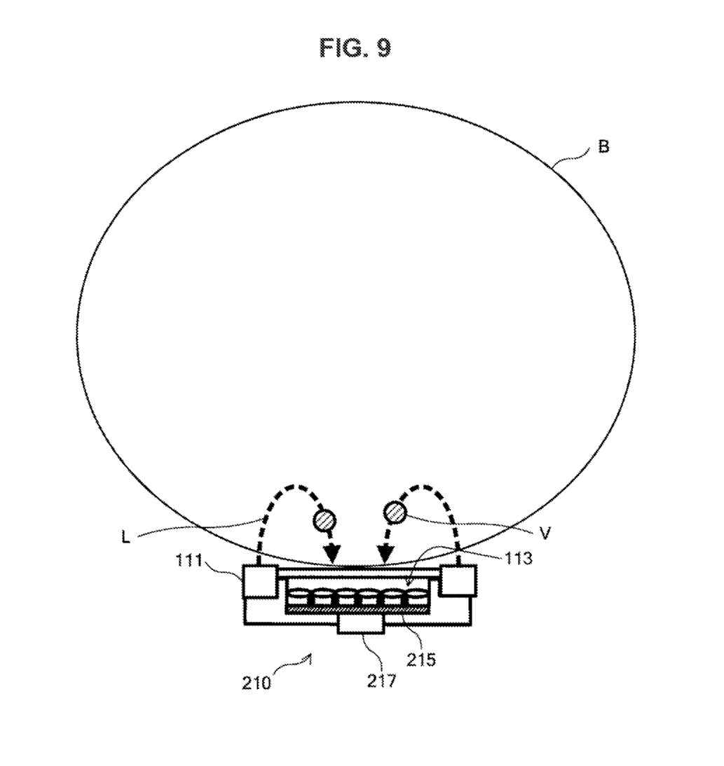

Next, with reference to FIG. 9, a second embodiment of the present disclosure will be described. In a measurement device according to the present embodiment, a configuration of a measurement unit 210 is different from that of the measurement unit 110 according to the first embodiment described above, but other constituent elements are substantially the same, and thus detailed description other than that of the measurement unit 210 will be omitted.

Referring to FIG. 9, the measurement unit 210 includes the light source unit 111, the microlens array 113, an image sensor 215, and an optical detector 217. The light source unit 111 and the microlens array 113 are the same constituent elements as those in the first embodiment.

The image sensor 215 only acquires captured images and does not acquire distribution of a light amount of the measurement light L, unlike the image sensor 115 of the first embodiment. Thus, the image sensor 215 may not necessarily deal with light of a wide wavelength band. In addition, as will be described below, distribution of the light amount of the measurement light L is acquired by the optical detector 217 provided on the side opposite to the side of the image sensor 215 as viewed from the living body B in the present embodiment. For this reason, in the image sensor 215, a material such as silicon that transmits light having a wavelength that is a detection target of the optical detector 217 is used.

The optical detector 217 is provided on an inner side of the image sensor 215 as viewed from the living body B to acquire the distribution of the light amount of the measurement light L. The optical detector 217 preferably deals with light having a wide wavelength band using a material, for example, indium gallium arsenide (InGaAs) or the like.

In the second embodiment of the present disclosure described above, the optical detector 217 that detects the measurement light L is provided separately from the image sensor 215 that acquires captured images of the living body B. Thus, in the present embodiment, the image sensor 215 may not be set to deal with a wide band.

### 3. Third Embodiment

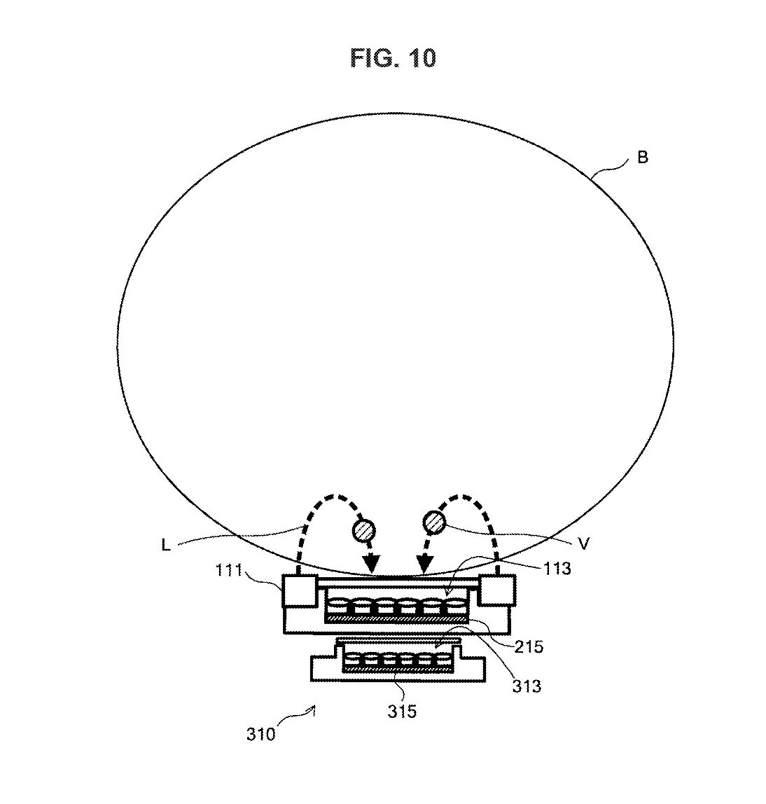

Next, with reference to FIG. 10, a third embodiment of the present disclosure will be described. In a measurement device according to the present embodiment, a configuration of a measurement unit 310 is different from that of the measurement unit 110 according to the first embodiment described above, but other constituent elements are substantially the same, and thus detailed description other than that of the measurement unit 310 will be omitted.

Referring to FIG. 10, the measurement unit 310 includes the light source unit 111, microlens arrays 113 and 313, and image sensors 215 and 315. The light source unit 111 and the microlens array 113 are the same constituent elements as those in the first embodiment. The image sensor 215 is the same constituent element as that in the second embodiment.

The microlens array 313 and the image sensor 315 are used instead of the optical detector 217 of the second embodiment. In other words, the microlens array 313 and the image sensor 315 are provided on an inner side of the image sensor 215 as viewed from the living body B. In other words, the microlens array 113 and the image sensor 215 are superimposed with the microlens array 313 and the image sensor 315 in a direction in which the measurement light L is discharged.

As the optical detector 217 described above, the image sensor 315 preferably deals with light having a wide wavelength band using a material, for example, indium gallium arsenide (InGaAs) or the like in order to acquire distribution of a light amount of the measurement light L. Note that the area of the microlens array 313 and the image sensor 315 may be smaller than the area of the microlens array 113 and the image sensor 215.

In the third embodiment of the present disclosure described above, the other microlens array 313 and the other image sensor 315 are provided separately from the image sensor 215 that acquires the captured images of the living body B. Accordingly, in addition to the fact that the image sensor 215 may not deal with a wide band, the measurement unit 110 can be made thin.

### 4. Fourth Embodiment

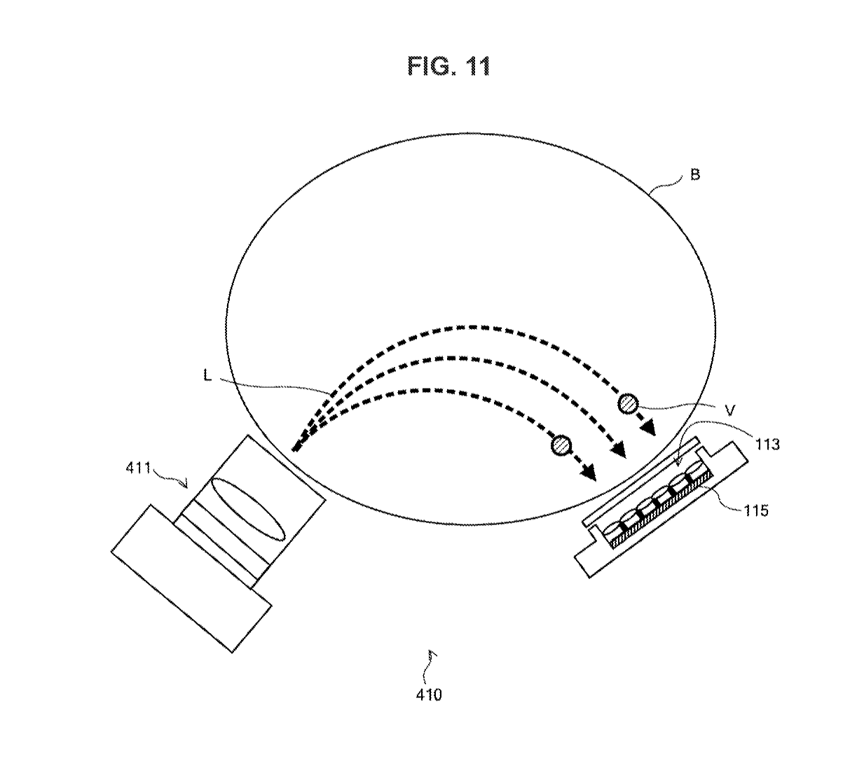

Next, with reference to FIG. 11, a fourth embodiment of the present disclosure will be described. In a measurement device according to the present embodiment, a configuration of a measurement unit 410 is different from that of the measurement unit 110 according to the first embodiment described above, but other constituent elements are substantially the same, and thus detailed description other than that of the measurement unit 410 will be omitted.

Referring to FIG. 11, the measurement unit 410 includes a light source unit 411, the microlens array 113, and the image sensors 115. The microlens array 113 and the image sensor 115 are the same constituent elements as those in the first embodiment.

The light source unit 411 radiates the measurement light L having a predetermined wavelength to the living body B. The light source unit 411 includes, for example, an LED array, a polarization filter, an object lens, and the like. The light source unit 411 is the same as the light source unit 111 according to the first embodiment in that an emission plane of the measurement light L is disposed to face the living body B, but is different from the light source unit 111 in that it is provided to be isolated from the microlens array 113.

As shown in the drawing, in the present embodiment, the measurement light L radiated from the light source unit 411 and then incident on the microlens array 113 passes through a wider range of the inside of the living body B. In other words, the measurement unit 410 can acquire measurement data in the wider range of the inside of the living body B. Thus, a person to be measured, for example, can measure a blood component of the vein V regardless of a position and an orientation of the living body B placed on the measurement unit 410.

### 5. Fifth Embodiment

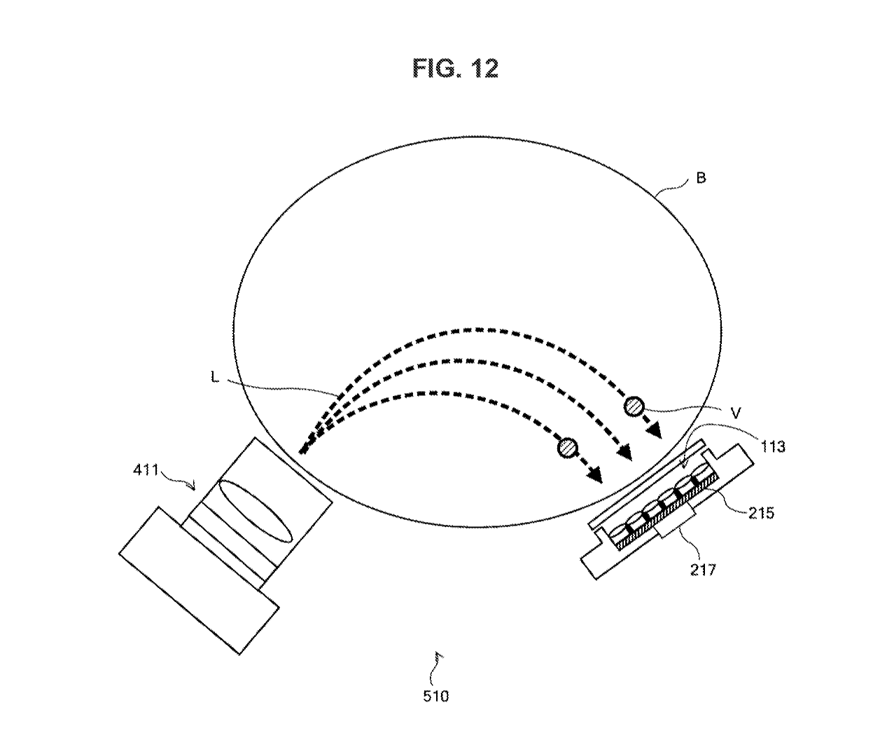

Next, with reference to FIG. 12, a fifth embodiment of the present disclosure will be described. In a measurement device according to the present embodiment, a configuration of a measurement unit 510 is different from that of the measurement unit 110 according to the first embodiment described above, but other constituent elements are substantially the same, and thus detailed description other than that of the measurement unit 510 will be omitted.

Referring to FIG. 12, the measurement unit 510 includes the light source unit 411, the microlens array 113, the image sensor 215, and the optical detector 217. The light source unit 411 is the same constituent element as that in the fourth embodiment. The microlens array 113, the image sensor 215, and the optical detector 217 are the same constituent elements as those in the second embodiment.

As described above, the present embodiment is a combination of the second embodiment and the fourth embodiment described above. Thus, in addition to the fact that measurement data can be acquired in a wider range of the inside of the living body B, the image sensor 215 may not deal with a wide band.

### 6. Sixth Embodiment

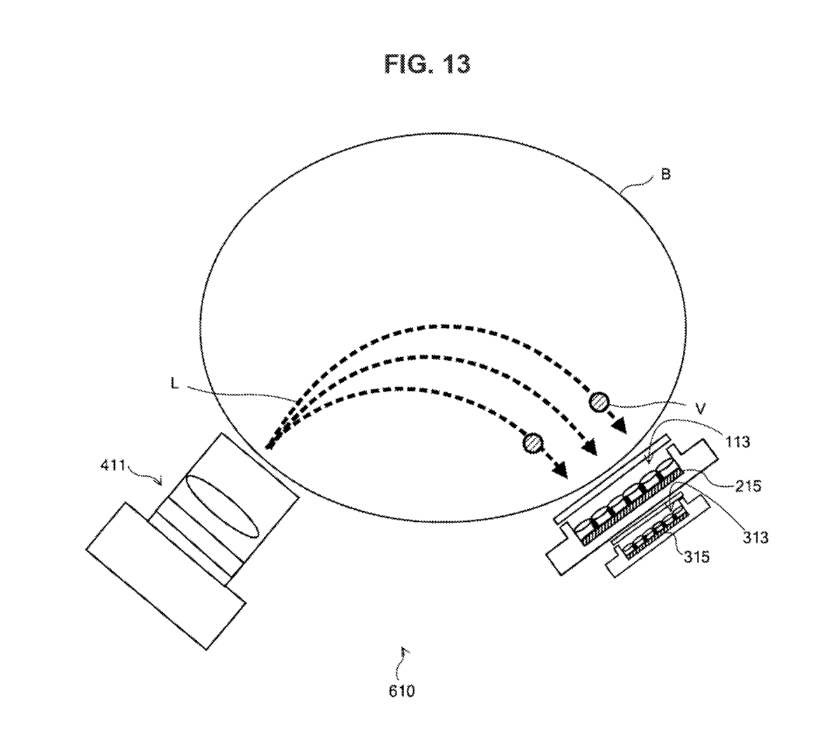

Next, with reference to FIG. 13, a sixth embodiment of the present disclosure will be described. In a measurement device according to the present embodiment, a configuration of a measurement unit 610 is different from that of the measurement unit 110 according to the first embodiment described above, but other constituent elements are substantially the same, and thus detailed description other than that of the measurement unit 610 will be omitted.

Referring to FIG. 13, the measurement unit 610 includes the light source unit 411, the microlens arrays 113 and 313, and the image sensors 215 and 315. The light source unit 411 is the same constituent element as that in the fourth embodiment. The microlens arrays 113 and 313, and the image sensors 215 and 315 are the same constituent elements as those in the third embodiment.

As described above, the present embodiment is a combination of the third embodiment and the fourth embodiment described above. Thus, in addition to the fact that measurement data can be acquired in a wider range of the inside of the living body B, the image sensor 215 may not deal with a wide band, and the measurement unit 610 can be made thin.

### (7. Supplement)

As described above, in the embodiments of the present disclosure, a blood component in a vein can be accurately measured through optical measurement. Accordingly, the same measurement as analysis of a blood component of a vein measured in hospitals can be realized in a non-invasive manner. In addition, analysis of a blood component is possible through calibration at a lower frequency than analysis of a body fluid of the past. In addition, by eliminating influence caused by a skin tissue or the like, the influence on a measurement result attributable to an individual difference can be reduced. In addition, since a change in a blood component in venous blood caused by influence of a meal or the like is shown more slightly than that in arterial blood, stable measurement is possible.

### (Hardware Configuration)

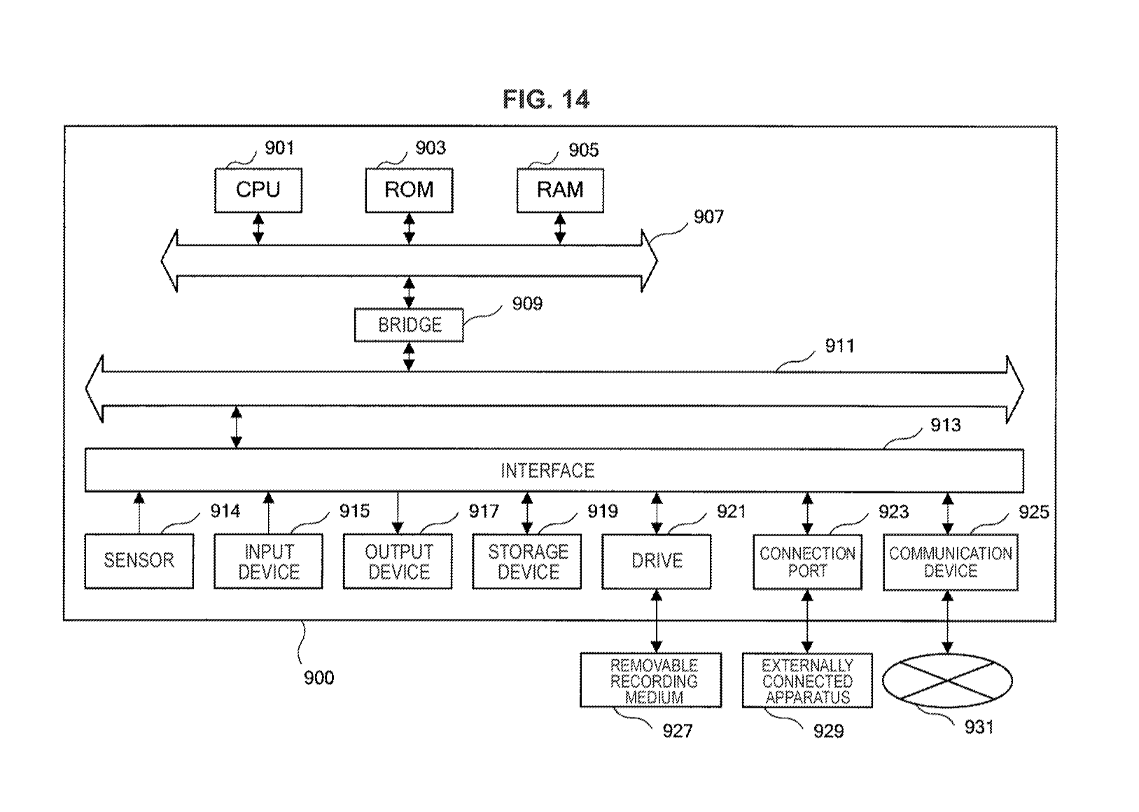

Lastly, the hardware configuration of an information processing apparatus 900, which is capable of realizing the measurement device according to an embodiment of the present disclosure, will be described in detail with reference to FIG. 14. FIG. 14 is a block diagram for illustrating the hardware configuration of the information processing apparatus 900 according to the embodiment of the present disclosure.

The information processing apparatus 900 mainly includes a CPU 901, a ROM 903, and a RAM 905. Furthermore, the information processing apparatus 900 also includes a host bus 907, a bridge 909, an external bus 911, an interface 913, a sensor 914, an input device 915, an output device 917, a storage device 919, a drive 921, a connection port 923, and a communication device 925.

The CPU 901 serves as an arithmetic processing device and a control device, and controls the overall operation or a part of the operation of the information processing apparatus 900 according to various programs recorded in the ROM 903, the RAM 905, the storage device 919, or a removable recording medium 927. The ROM 903 stores programs, operation parameters, and the like used by the CPU 901. The RAM 905 primarily stores programs that the CPU 901 uses and parameters and the like varying as appropriate during the execution of the programs. These are connected with each other via the host bus 907 configured from an internal bus such as a CPU bus or the like.

The host bus 907 is connected to the external bus 911 such as a PCI (Peripheral Component Interconnect/Interface) bus via the bridge 909.

The sensor 914 is detecting means for detecting biological information unique to a user or various types of information to be used to acquire such biological information. This sensor 914 includes, for example, various image sensors such as a CCD (Charge Coupled Device) or a CMOS (Complementary Metal Oxide Semiconductor) and the like. In addition, the sensor 914 may further have optics such as a lens to be used to image an organism site or a light source and the like. The sensor 914 may also be a microphone and the like for acquiring sound and the like. Note that in addition to those mentioned above, the sensor 914 may also include various measuring instruments such as a thermometer, an illuminance meter, a hygrometer, a speedometer, an accelerometer, and the like.

The input device 915 is operation means operated by a user, such as a mouse, a keyboard, a touch panel, buttons, a switch and a lever. Also, the input device 915 may be a remote controlling means (a so-called remote controller) using, for example, infrared light or other radio waves, or may be an externally connected apparatus 929 such as a mobile phone or a PDA conforming to the operation of the information processing apparatus 900. Furthermore, the input device 915 generates an input signal based on, for example, information which is input by a user with the above operation means, and is configured from an input control circuit for outputting the input signal to the CPU 901. The user of the information processing apparatus 900 can input various data to the information processing apparatus 900 and can instruct the information processing apparatus 900 to perform processing by operating this input apparatus 915.

The output device 917 is configured from a device capable of visually or audibly notifying user of acquired information. Examples of such device include display devices such as a CRT display device, a liquid crystal display device, a plasma display device, an EL display device and lamps, audio output devices such as a speaker and a headphone, a printer, a mobile phone, a facsimile machine, and the like. For example, the output device 917 outputs a result obtained by various processing performed by the information processing apparatus 900. More specifically, the display device displays, in the form of text or images, a result obtained by various processes performed by the information processing apparatus 900. On the other hand, the audio output device converts an audio signal such as reproduced audio data and sound data into an analog signal, and outputs the analog signal.

The storage device 919 is a device for storing data configured as an example of a storage unit of the information processing apparatus 900 and is used to store data. The storage device 919 is configured from, for example, a magnetic storage device such as a HDD (Hard Disk Drive), a semiconductor storage device, an optical storage device, or a magneto-optical storage device. This storage device 919 stores programs to be executed by the CPU 901, various data, and various data obtained from the outside.

The drive 921 is a reader/writer for recording medium, and is embedded in the information processing apparatus 900 or attached externally thereto. The drive 921 reads information recorded in the attached removable recording medium 927 such as a magnetic disk, an optical disc, a magneto-optical disc, or a semiconductor memory, and outputs the read information to the RAM 905. Furthermore, the drive 921 can write in the attached removable recording medium 927 such as a magnetic disk, an optical disc, a magneto-optical disc, or a semiconductor memory. The removable recording medium 927 is, for example, a DVD medium, an HD-DVD medium, or a Blu-ray medium. The removable recording medium 927 may be a CompactFlash (CF; registered trademark), a flash memory, an SD memory card (Secure Digital Memory Card), or the like. Alternatively, the removable recording medium 927 may be, for example, an IC card (Integrated Circuit Card) equipped with a non-contact IC chip or an electronic appliance.

The connection port 923 is a port for allowing devices to directly connect to the information processing apparatus 900. Examples of the connection port 923 include a USB (Universal Serial Bus) port, an IEEE1394 port, a SCSI (Small Computer System Interface) port, and the like. Other examples of the connection port 923 include an RS-232C port, an optical audio terminal, an HDMI (High-Definition Multimedia Interface) port, and the like. By the externally connected apparatus 929 connecting to this connection port 923, the information processing apparatus 900 directly obtains various data from the externally connected apparatus 929 and provides various data to the externally connected apparatus 929.

The communication device 925 is a communication interface configured from, for example, a communication device for connecting to a communication network 931. The communication device 925 is, for example, a wired or wireless LAN (Local Area Network), Bluetooth (registered trademark), a communication card for WUSB (Wireless USB), or the like. Alternatively, the communication device 925 may be a router for optical communication, a router for ADSL (Asymmetric Digital Subscriber Line), a modem for various communications, or the like. This communication device 925 can transmit and receive signals and the like in accordance with a predetermined protocol such as TCP/IP on the Internet and with other communication devices, for example. The communication network 931 connected to the communication device 925 is configured from a network and the like, which is connected via wire or wirelessly, and may be, for example, the Internet, a home LAN, infrared communication, radio wave communication, satellite communication, or the like.

Heretofore, an example of the hardware configuration capable of realizing the functions of the information processing apparatus 900 according to the embodiment of the present disclosure has been shown. Each of the constituent elements described above may be configured using a general-purpose material, or may be configured from hardware dedicated to the function of each constituent element. Accordingly, the hardware configuration to be used can be changed as appropriate according to the technical level at the time of carrying out the present embodiment.

The preferred embodiments of the present invention have been described above with reference to the accompanying drawings, whilst the present invention is not limited to the above examples, of course. A person skilled in the art may find various alterations and modifications within the scope of the appended claims, and it should be understood that they will naturally come under the technical scope of the present invention.

Additionally, the present technology may also be configured as below.

(1)

A measurement device including:

a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape;

a vein position specification unit configured to specify a position of a vein present inside the living body based on the captured image;

a vein depth specification unit configured to specify a depth of the vein based on the captured image; and

a blood component estimation unit configured to estimate a blood component of the vein based on information obtained from the detected measurement light using the position of the vein and the depth of the vein.

(2)

The measurement device according to (1), wherein the blood component estimation unit estimates a thickness of a body tissue present between a surface of the living body and the vein based on the depth of the vein, and then estimates the blood component of the vein excluding influence of the body tissue of the estimated thickness from information obtained from the measurement light detected in the position of the vein.

(3)

The measurement device according to (1) or (2), wherein the blood component estimation unit estimates the blood component of the vein by comparing information obtained from the measurement light detected in an adjacent area adjacent to the position of the vein to the information obtained from the measurement light detected in the position of the vein.

(4)

The measurement device according to (3), wherein the blood component estimation unit estimates the blood component of the vein by correcting the information obtained from the measurement light detected in the position of the vein using the information obtained from the measurement light detected in the adjacent area adjacent to the position of the vein.

(5)

The measurement device according to (4), wherein the blood component estimation unit extracts a time change component derived from beats of an artery present inside the living body from the information obtained from the measurement light detected in the adjacent area, and then estimates the blood component of the vein by excluding the time change component from the information obtained from the measurement light detected in the position of the vein.

(6)

The measurement device according to any one of (1) to (5),

wherein the measurement unit includes an image sensor configured to acquire the captured image,

wherein, in the image sensor, a plurality of light receiving elements are allocated to one of the light receiving lenses, and

wherein the vein depth specification unit specifies a depth of the vein based on parallax information extracted from the captured image acquired by the plurality of light receiving elements corresponding to the same light receiving lens.

(7)

The measurement device according to (6), wherein the vein depth specification unit extracts the parallax information from the captured image using the technique of Light Field Photography.

(8)

The measurement device according to any one of (1) to (7), wherein the blood component estimation unit performs calibration on an estimation result of the blood component based on a blood component measured through prior blood sampling.

(9)

The measurement device according to any one of (1) to (8), wherein the measurement unit includes an image sensor configured to detect the discharged measurement light and to acquire the captured image.

(10)

The measurement device according to any one of (1) to (8), wherein the measurement unit includes a detector configured to detect the discharged measurement light and an image sensor configured to acquire the captured image.

(11)

The measurement device according to (10), wherein the measurement unit includes a lens array separate from the lens array and an image sensor separate from the image sensor as the detector.

(12)

The measurement device according to (10) or (11), wherein the image sensor and the detector are superimposed in a direction in which the measurement light is discharged, and the image sensor transmits at least part of the measurement light.

(13)

The measurement device according to any one of (1) to (12),

wherein the measurement unit includes a light source unit configured to radiate the measurement light, and

wherein the light source unit is provided at an edge of the lens array.

(14)

The measurement device according to any one of (1) to (12),

wherein the measurement unit includes a light source unit configured to radiate the measurement light, and

wherein the light source unit is provided to be isolated from the lens array.

(15)

A measurement method including;

radiating measurement light having a predetermined wavelength to at least a part of a living body, detecting the measurement light scattered inside the living body and then discharged from a surface of the living body, and acquiring a captured image of the living body by collecting the discharged measurement light using a lens array having a plurality of light receiving lenses arranged in an array shape;

specifying a position of a vein present inside the living body based on the captured image;

specifying a depth of the vein based on the captured image; and

estimating a blood component of the vein based on information obtained from the detected measurement light using the position of the vein and the depth of the vein.

(16)

A program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute:

a vein position specification function of specifying a position of a vein present inside the living body based on the captured image;

a vein depth specification function of specifying a depth of the vein based on the captured image; and

a blood component estimation function of estimating a blood component of the vein based on information obtained from the detected measurement light using the position of the vein and the depth of the vein.

(17)

A computer-readable recording medium on which a program is recorded, the program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute:

a vein position specification function of specifying a position of a vein present inside the living body based on the captured image;

a vein depth specification function of specifying a depth of the vein based on the captured image; and

a blood component estimation function of estimating a blood component of the vein based on information obtained from the detected measurement light using the position of the vein and the depth of the vein.

(18)

A measurement device including:

a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape;

a vein position specification unit configured to specify a position of a vein present inside the living body based on the captured image;

a vein depth specification unit configured to specify a depth of the vein based on the captured image; and

a blood component estimation unit configured to estimate a thickness of a body tissue present between a surface of the living body and the vein based on the depth of the vein, to exclude influence of the body tissue of the estimated thickness from information obtained from the measurement light detected in the position of the vein, to extract a time change component derived from beats of an artery present inside the living body from information obtained from the measurement light detected in an adjacent area adjacent to the position of the vein, and to estimate a blood component of the vein by further eliminating the time change component from the information obtained from the measurement light detected in the position of the vein.

(19)

A measurement method including:

radiating measurement light having a predetermined wavelength to at least a part of a living body, detecting the measurement light scattered inside the living body and then discharged from a surface of the living body, and acquiring a captured image of the living body by collecting the discharged measurement light using a lens array having a plurality of light receiving lenses arranged in an array shape;

specifying a position of a vein present inside the living body based on the captured image;

specifying a depth of the vein based on the captured image; and

estimating a thickness of a body tissue present between a surface of the living body and the vein based on the depth of the vein, excluding influence of the body tissue of the estimated thickness from information obtained from the measurement light detected in the position of the vein, extracting a time change component derived from beats of an artery present inside the living body from information obtained from the measurement light detected in an adjacent area adjacent to the position of the vein, and estimating a blood component of the vein by further eliminating the time change component from the information obtained from the measurement light detected in the position of the vein.

(20)

A program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute:

a vein position specification function of specifying a position of a vein present inside the living body based on the captured image;

a vein depth specification function of specifying a depth of the vein based on the captured image; and

a blood component estimation function of estimating a thickness of a body tissue present between a surface of the living body and the vein based on the depth of the vein, excluding influence of the body tissue of the estimated thickness from information obtained from the measurement light detected in the position of the vein, extracting a time change component derived from beats of an artery present inside the living body from information obtained from the measurement light detected in an adjacent area adjacent to the position of the vein, and estimating a blood component of the vein by further eliminating the time change component from the information obtained from the measurement light detected in the position of the vein.

### REFERENCE SIGNS LIST

100 measurement device
110 measurement unit
140 vein position specification unit
150 vein depth specification unit
160 blood component estimation unit
180 storage unit

1. A measurement device comprising:
a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape;
a vein position specification unit configured to specify a position of a vein present inside the living body based on the captured image;
a vein depth specification unit configured to specify a depth of the vein based on the captured image; and
a blood component estimation unit configured to estimate a blood component of the vein based on information obtained from the detected measurement light using the position of the vein and the depth of the vein.

2. The measurement device according to claim 1, wherein the blood component estimation unit estimates a thickness of a body tissue present between a surface of the living body and the vein based on the depth of the vein, and then estimates the blood component of the vein excluding influence of the body tissue of the estimated thickness from information obtained from the measurement light detected in the position of the vein.

3. The measurement device according to claim 1, wherein the blood component estimation unit estimates the blood component of the vein by comparing information obtained from the measurement light detected in an adjacent area adjacent to the position of the vein to the information obtained from the measurement light detected in the position of the vein.

4. The measurement device according to claim 3, wherein the blood component estimation unit estimates the blood component of the vein by correcting the information obtained from the measurement light detected in the position of the vein using the information obtained from the measurement light detected in the adjacent area adjacent to the position of the vein.

5. The measurement device according to claim 4, wherein the blood component estimation unit extracts a time change component derived from beats of an artery present inside the living body from the information obtained from the measurement light detected in the adjacent area, and then estimates the blood component of the vein by excluding the time change component from the information obtained from the measurement light detected in the position of the vein.

6. The measurement device according to claim 1,
wherein the measurement unit includes an image sensor configured to acquire the captured image,
wherein, in the image sensor, a plurality of light receiving elements are allocated to one of the light receiving lenses, and
wherein the vein depth specification unit specifies a depth of the vein based on parallax information extracted from the captured image acquired by the plurality of light receiving elements corresponding to the same light receiving lens.

7. The measurement device according to claim 6, wherein the vein depth specification unit extracts the parallax information from the captured image using the technique of Light Field Photography.

8. The measurement device according to claim 1, wherein the blood component estimation unit performs calibration on an estimation result of the blood component based on a blood component measured through prior blood sampling.

9. The measurement device according to claim 1, wherein the measurement unit includes an image sensor configured to detect the discharged measurement light and to acquire the captured image.

10. The measurement device according to claim 1, wherein the measurement unit includes a detector configured to detect the discharged measurement light and an image sensor configured to acquire the captured image.

11. The measurement device according to claim 10, wherein the measurement unit includes a lens array separate from the lens array and an image sensor separate from the image sensor as the detector.

12. The measurement device according to claim 10, wherein the image sensor and the detector are superimposed in a direction in which the measurement light is discharged, and the image sensor transmits at least part of the measurement light.

13. The measurement device according to claim 1,
wherein the measurement unit includes a light source unit configured to radiate the measurement light, and
wherein the light source unit is provided at an edge of the lens array.

14. The measurement device according to claim 1,
wherein the measurement unit includes a light source unit configured to radiate the measurement light, and
wherein the light source unit is provided to be isolated from the lens array.

15. A measurement method comprising;
radiating measurement light having a predetermined wavelength to at least a part of a living body, detecting the measurement light scattered inside the living body and then discharged from a surface of the living body, and acquiring a captured image of the living body by collecting the discharged measurement light using a lens array having a plurality of light receiving lenses arranged in an array shape;
specifying a position of a vein present inside the living body based on the captured image;
specifying a depth of the vein based on the captured image; and
estimating a blood component of the vein based on information obtained from the detected measurement light using the position of the vein and the depth of the vein.

16. A program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute:
a vein position specification function of specifying a position of a vein present inside the living body based on the captured image;
a vein depth specification function of specifying a depth of the vein based on the captured image; and
a blood component estimation function of estimating a blood component of the vein based on information obtained from the detected measurement light using the position of the vein and the depth of the vein.

17. A computer-readable recording medium on which a program is recorded, the program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute:
a vein position specification function of specifying a position of a vein present inside the living body based on the captured image;
a vein depth specification function of specifying a depth of the vein based on the captured image; and
a blood component estimation function of estimating a blood component of the vein based on information obtained from the detected measurement light using the position of the vein and the depth of the vein.

18. A measurement device comprising:
a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape;
a vein position specification unit configured to specify a position of a vein present inside the living body based on the captured image;
a vein depth specification unit configured to specify a depth of the vein based on the captured image; and
a blood component estimation unit configured to estimate a thickness of a body tissue present between a surface of the living body and the vein based on the depth of the vein, to exclude influence of the body tissue of the estimated thickness from information obtained from the measurement light detected in the position of the vein, to extract a time change component derived from beats of an artery present inside the living body from information obtained from the measurement light detected in an adjacent area adjacent to the position of the vein, and to estimate a blood component of the vein by further eliminating the time change component from the information obtained from the measurement light detected in the position of the vein.

19. A measurement method comprising:
radiating measurement light having a predetermined wavelength to at least a part of a living body, detecting the measurement light scattered inside the living body and then discharged from a surface of the living body, and acquiring a captured image of the living body by collecting the discharged measurement light using a lens array having a plurality of light receiving lenses arranged in an array shape;
specifying a position of a vein present inside the living body based on the captured image;
specifying a depth of the vein based on the captured image; and
estimating a thickness of a body tissue present between a surface of the living body and the vein based on the depth of the vein, excluding influence of the body tissue of the estimated thickness from information obtained from the measurement light detected in the position of the vein, extracting a time change component derived from beats of an artery present inside the living body from information obtained from the measurement light detected in an adjacent area adjacent to the position of the vein, and estimating a blood component of the vein by further eliminating the time change component from the information obtained from the measurement light detected in the position of the vein.

20. A program causing a computer capable of communicating with a measurement unit configured to radiate measurement light having a predetermined wavelength to at least a part of a living body, to detect the measurement light scattered inside the living body and then discharged from a surface of the living body, and to acquire a captured image of the living body by collecting the discharged measurement light using a lens array having a plurality of light receiving lenses arranged in an array shape to execute:
a vein position specification function of specifying a position of a vein present inside the living body based on the captured image;
a vein depth specification function of specifying a depth of the vein based on the captured image; and
a blood component estimation function of estimating a thickness of a body tissue present between a surface of the living body and the vein based on the depth of the vein, excluding influence of the body tissue of the estimated thickness from information obtained from the measurement light detected in the position of the vein, extracting a time change component derived from beats of an artery present inside the living body from information obtained from the measurement light detected in an adjacent area adjacent to the position of the vein, and estimating a blood component of the vein by further eliminating the time change component from the information obtained from the measurement light detected in the position of the vein.
